(19) United States
(12) Patent Application Publication
(10) Pub. No.: US 2026/0256713 A1
(43) Pub. Date: Sep. 3, 2026

(54) USE OF RESVERATROL IN PREPARATION OF PRODUCT FOR PREVENTING AND/OR TREATING UNEXPLAINED RECURRENT SPONTANEOUS ABORTION

(71) Applicant: The First Affiliated Hospital of Anhui Medical University, Hefei (CN)

(72) Inventors: Huijuan Zou, Hefei (CN); Xingxing Han, Hefei (CN); Zhiguo Zhang, Hefei (CN); Beili Chen, Hefei (CN); Xiaofeng Xu, Hefei (CN); Yunxia Cao, Hefei (CN)

(21) Appl. No.: 19/182,443

(22) Filed: Apr. 17, 2025

(30) Foreign Application Priority Data
Mar. 3, 2025 (CN) 202510237679.4

(51) Int. Cl. A61K 31/05; A61P 15/06

(52) U.S. Cl. CPC A61K 31/05; A61P 15/06

(57) ABSTRACT

Disclosed is use of resveratrol in the preparation of a product for preventing and/or treating unexplained recurrent spontaneous abortion, which belongs to the technical field of biomedicines. According to the present disclosure, after a small dose of resveratrol is intragastrically administered to mice with unexplained recurrent spontaneous abortion, not only the embryo resorption rate is reduced, but also the inflammatory response of the mouse decidual tissues is improved, the Sirt1 level of the mouse decidual tissues is increased, and the occurrence and development of aseptic inflammation at the maternal-fetal interface of the mice with unexplained recurrent spontaneous abortion are prevented, thus the pregnancy rate of mice with unexplained recurrent spontaneous abortion is effectively protected and treated. The present disclosure provides a new approach and method for the treatment of unexplained recurrent spontaneous abortion.

### CROSS-REFERENCE TO RELATED APPLICATION

This application claims priority of Chinese Patent Application No. 202510237679.4, filed on Mar. 3, 2025, the entire contents of which are incorporated herein by reference.

### TECHNICAL FIELD

The present disclosure belongs to the technical field of biomedicines, and in particular relates to use of resveratrol in the preparation of a product for preventing and/or treatment of unexplained recurrent spontaneous abortion.

### BACKGROUND

Recurrent spontaneous abortion (RSA) is a common complication among women of childbearing age. It refers to two or more clinical pregnancy failures, with an incidence rate of up to 1-5%. The etiology of RSA are diverse, including chromosomal abnormalities, age, antiphospholipid syndrome, uterine malformations, thrombosis, hormonal or metabolic disorders, infection, autoimmunity, sperm quality and lifestyle, etc. In 50-75% of cases involving patients with recurrent spontaneous abortion (RSA), the cause remains unidentified, a condition known as unexplained recurrent spontaneous abortion (URSA). Given that the etiology and pathogenesis of URSA have not been fully understood, there is currently no universally accepted international diagnostic standard for the condition. In addition, the therapeutic method for URSA has not been determined, and there is a scarcity of targeted and effective treatment regimens. Currently, targeted preventive treatment is primarily conducted based on the etiology of the disease. For women at high risk, commonly used intervention measures include active lymphocyte immunotherapy, intravenous immunoglobulin, glucocorticoids, cyclophosphamide A, G-CSF, progesterone, TNF-α inhibitors, and anticoagulant therapy. However, the routine use of these treatments is not recommended in existing guidelines or expert consensus on miscarriage. Therefore, the research on URSA is still very important. In-depth understanding of the causes and pathogenic mechanisms of URSA and the development of new targeted treatments are of great significance for reducing the incidence of URSA and promoting healthy pregnancy.

In recent years, numerous studies have shown that the occurrence of URSA is closely associated with the imbalance of maternal and fetal immune tolerance. Compared with normal pregnancies, patients with URSA exhibit disruption of the maternal-fetal interface, irregular cell arrangement, a higher rate of nuclear rupture, and increased infiltrating cells in the chorionic villi and decidua.

Resveratrol (3,4,5-trihydroxy-trans-stilbene, RES) is a common non-flavonoid polyphenol compound and is widely distributed in various plants such as the root of Polygonum cuspidatum, peanuts, and grape skins. It can be extracted and purified by supercritical fluid extraction, Soxhlet extraction, enzymatic hydrolysis, etc. Studies have shown that it has multiple pharmacological effects such as anti-inflammatory, antioxidant, anti-aging, and anti-tumor. It has been confirmed to be a natural compound that can effectively activate NAD⁺-dependent deacetylases-Sirtuins family proteins. Presently, SIRT1 is the most studied mammalian sirtuin. It is a NAD⁺ (nicotinamide adenine dinucleotide)-dependent histone deacetylase that is involved in regulating metabolism, aging, cell survival, autophagy, etc. Sirt1 can activate a variety of proteins and transcription factors (such as HMGB1, PGC-1α, NF-κB, NLRP-3, etc.) through deacetylation, promote mitochondrial biogenesis, and participate in many physiological and pathological processes, such as oxidative stress and cell apoptosis. Sirt1 plays an important regulatory role in inflammatory response. Resveratrol has been shown to significantly improve inflammatory-related diseases. It not only significantly reduces the levels of pro-inflammatory markers IL-6 and TNF-α in the liver of mice with non-alcoholic fatty liver induced by a high-fat diet, but also inhibits NLRP3 activation-induced cell pyroptosis, exerting a positive protective effect in liver functions. In addition, resveratrol can protect the myocardium of mice with endotoxemia by mediating the miR-149/HMGB1 axis and regulating the ferroptosis pathway. Because of these characteristics, resveratrol has become a valuable therapeutic tool for the treatment of various diseases associated with Sirt1.

There are also reports on the roles of resveratrol in pregnancy-related diseases. The study by Wang et al. showed that supplementation with 40 mg/kg resveratrol may promote the proliferation and antioxidant ability of mammary glandular cells through mitochondrial autophagy and regulation of the intestinal microbiota of pregnant mice. In addition, resveratrol can improve uterine mitochondrial function in mice fed a long-term high-fat diet, thereby improving obesity-related adverse pregnancy outcomes. However, there are no reports on the clinical use of resveratrol for the treatment of unexplained recurrent spontaneous abortion.

### SUMMARY

The present disclosure provides use of resveratrol in the preparation of a product for preventing and/or treating unexplained recurrent spontaneous abortion. The chemical formula of resveratrol is C₁₄H₁₂O_{3,} and the CAS registration number is 501-36-0.

Preferably, the product is a drug.

More preferably, the resveratrol is the only active ingredient in the drug.

Still more preferably, the drug further contains medically acceptable excipients.

Still more preferably, a dosage form of the drug is injection, solution, emulsion, suspension, suppository, ointment, cream, spray, drop, powder, granule, dissolved medicine, capsule, pill, tablet, patch or sustained-release preparation.

The present disclosure further provides use of a composition in the preparation of a product for preventing and/or treating unexplained recurrent spontaneous abortion, where the composition contains resveratrol.

Preferably, the product is a drug.

More preferably, the drug further contains medically acceptable excipients.

Even more preferably, a dosage form of the drug includes injection, solution, emulsion, suspension, suppository, ointment, cream, spray, drop, powder, granule, dissolved medicine, capsule, pill, tablet, patch or sustained-release preparation.

Compared with the existing technology, the present disclosure has the following beneficial effects:
The present disclosure relates to use of resveratrol in the preparation of drugs for preventing and treating unexplained recurrent spontaneous abortion. According to the present disclosure, after a small dose of resveratrol is intragastrically administered to mice with unexplained recurrent spontaneous abortion, not only the embryo resorption rate is reduced, but also the inflammatory response of the mouse decidual tissues is improved, the Sirt1 level of the mouse decidual tissues is increased, and the occurrence and development of aseptic inflammation at the maternal-fetal interface of the mice with unexplained recurrent spontaneous abortion are prevented, thus the pregnancy rate of mice with unexplained recurrent spontaneous abortion is effectively protected and treated.

### BRIEF DESCRIPTION OF THE DRAWINGS

FIG. 1A shows that the intervention group in Example 1 was given a small dose of resveratrol by gavage before and after cage combination, and the control group and URSA group were given the same dose of solvent by gavage. The mice were dissected and tissues were obtained on the 14th day of pregnancy. The left and right uterine horns, fetuses, and placentas of normal pregnant mice, aborted mice, and mice treated with drugs at different doses (25, 50, and 100 mg/kg/d).

FIG. 1B is a statistical graph of the number of embryos in each group showing that the intervention group in Example 1 was given a small dose of resveratrol by gavage before and after cage combination, and the control group and URSA group were given the same dose of solvent by gavage. The mice were dissected and tissues were obtained on the 14th day of pregnancy.

FIG. 2A shows the pathological conditions of the decidual tissues of normal pregnant mice, aborted mice, and mice treated with the drugs at a dose of 50 mg/kg/d in Example 1. H&E staining results of decidual tissues of normal pregnant mice, aborted mice, and mice treated with resveratrol.

FIG. 2B shows the pathological conditions of the decidual tissues of normal pregnant mice, aborted mice, and mice treated with the drugs at a dose of 50 mg/kg/d in Example 1.The cell infiltration in the decidual tissues of the three groups of mice was quantitatively evaluated by counting, with a scale bar of 50 μm. *** represents a significant difference among the groups (P<0.001), and ** represents a significant difference among the groups (P<0.01).

FIG. 3A shows the expression of Sirt1 protein in the decidual tissues of normal pregnant mice, aborted mice and mice treated with the drug at a dose of 50 mg/kg/d in Example 1. Immunohistochemistry was used to detect the expression and distribution of Sirt1 protein in the decidual tissues of normal pregnant mice, aborted mice and mice treated with resveratrol.

FIG. 3B shows the expression of Sirt1 protein in the decidual tissues of normal pregnant mice, aborted mice and mice treated with the drug at a dose of 50 mg/kg/d in Example 1. ImageJ was used to quantitatively evaluate the average optical density value of Sirt1 protein expression in the decidual tissues of mice in the three groups, with a scale bar of 50 μm. * represents a significant difference among the groups (P<0.05).

### DETAILED DESCRIPTION

In the following embodiments, the resveratrol liquid preparation was prepared by dissolving resveratrol powder (MedChemExpress) in a solvent of PEG300 and saline (1:1).

### Example 1

The experiment of the present disclosure of low-dose resveratrol protecting unexplained recurrent spontaneous abortion based on Sirt1 molecule was as follows.

(2) The implementation of the technology of low-dose resveratrol targeting Sirt1 molecule to protect unexplained recurrent spontaneous abortion was as follows.

Female CBA/J mice, male DBA/2J mice and male BALB/c mice aged 6-8 weeks were purchased from Beijing Huafukang Biotechnology Co., Ltd. All mice were adaptively raised in a standard environment with a constant temperature of 21-23° C. and a humidity of 50-60% for one week and were given autoclaved water and food.

After one week of adaptive feeding, all female CBA/J mice were randomly divided into three groups, including the control group (CBA/J♀×BALB/c♂), the model group (URSA group, CBA/J♀×DBA/ 2J♂) and the treatment group (URSA+RES group (CBA/J♀×DBA/ 2J♂), with 8 mice in each group. Then, the right ear of each female mouse was marked with an ear tag, and adaptive feeding was performed again for one week. (Modeling Group: CBA/J female mice were prone to miscarriage when they mated with DBA/2J male mice, and the reason was unknown, so they were often used as URSA mouse models. During pregnancy, bleeding could be observed at the vaginal opening of female mice, and when mice were killed on the 14th day of pregnancy, resorption of the fetus could be observed, which was a sign of miscarriage). Subsequently, the female mice in the treatment group were given resveratrol by gavage for prevention at a dose of 25, 50, and 100 mg/kg/d at 6:00 p.m. The mice in the control group and the model group were given the same dose of solvent by gavage at the same time, and all drinking water was replaced every two days. After 7 consecutive days of gavage, at 6:00 p.m., every two female CBA/J mice were caged with one male BALB/c mouse in the control group; and in the model group, every two female CBA/J mice were caged with one male DBA/2J mouse; and in the treatment group, every two female CBA/J mice were caged with one male DBA/ 2J mouse. Before 8:00 a.m., the next morning, all female mice were examined and separated into cages, and female mice with vaginal plugs detected were recorded as gestational day 0(GD0 ). Subsequently, the food intake, activity, vaginal bleeding, and body weight of mice in each group were observed and recorded every day. On GD7, all female mice stopped gavage. All mice were euthanized on gestational day 14 (GD14), and venous blood was collected from the eyeballs and placed at room temperature for two hours. The blood was then centrifuged at 3,000 rpm for 10 minutes at 4 degrees Celsius, and the supernatant was aspirated and placed in a −80° C. refrigerator for later use. At the same time, the mouse uterus, embryos, placenta, and decidua were separated, and all tissues were fixed with 4% paraformaldehyde and stored at −80° C. The total number of fetuses and the number of live fetuses were counted. Absorbed fetuses were small and black with necrosis and hemorrhage. The embryo resorption rate was calculated as the number of absorbed embryos/total number of embryos×100%.

(2) The protective effect of resveratrol on mice with unexplained recurrent spontaneous abortion was tested as follows:

### Detection on Fetal Absorption

When the mice were raised to the 14th day of pregnancy, the pregnant mice were killed by cervical dislocation, their eyeballs were quickly removed to collect blood, and the abdominal cavity was opened to remove the uterus and take pictures. Then the fetus, placenta, absorption fetus and decidua were separated, and the number of viable fetuses and absorbed fetuses was counted.

### H&E Staining

After the mouse decidua tissue was fixed with 4% paraformaldehyde for 24-48 hours, all samples were dehydrated with alcohol in a concentration gradient of 70%, 80%, 90%, and 100%, and then embedded in paraffin after immersion in a mixture of alcohol and xylene and xylene. The wax blocks were cut into 4-μm thick paraffin sections. The paraffin sections were immersed in xylene for 20 minutes, repeated once, then immersed in 100%, 90%, 80%, and 70% alcohol for 2 minutes, washed twice with distilled water, stained with hematoxylin for 3 minutes, observed under a microscope for appropriate color, rinsed with running water for 5 minutes, and then stained with eosin for 2 minutes. After dehydration with gradient alcohol, the staining effect was observed under a microscope, and then permeabilized with xylene for 30 minutes. All sections were then observed with resin sealing and a fully automatic high-resolution panoramic imaging analysis system.

### Detection of Sirt1 Protein Distribution in Decidual Tissues

The previously prepared mouse decidua paraffin sections were baked in an oven at 60° C. for 2 hours, then dewaxed with xylene and gradient alcohol, and then the sections were permeabilized with 2% Triton solution (Labgic Technology Co., Ltd., China, membrane permeabilizing agent for nuclear perforation) for 20 minutes, and then repaired with sodium citrate antigen retrieval solution. After blocking endogenous peroxidase (ZSGB BIO, China) for 10 minutes, the sections were incubated with Sirt1 (1:200 dilution) at 4° C. overnight, and then all samples were incubated with reaction enhancement solution (ZSGB BIO, China) for 20 minutes, washed, incubated with enhanced enzyme-labeled goat anti-mouse/rabbit IgG polymer (ZSGB BIO, China) for 20 minutes, and stained with a DAB color development kit (diaminobenzidine, ZSGB BIO, China). After the sections were blocked with resin, positive cells were observed under a fully automatic high-resolution panoramic imaging analysis system. DAB was a common substrate for horseradish peroxidase. Under the catalysis of horseradish peroxidase, DAB would produce a brown precipitate.

At present, experiments related to the present disclosure were completed in the Key Laboratory of Gamete and Reproductive Tract Abnormalities of the National Health Commission. The experimental results were as follows.

In the control group, there were 8 mice and 8 pregnant mice. The number of viable fetuses was 66, the number of absorbed fetuses was 4, and the embryo resorption rate was 5.7%.

In the model group, there were 8 mice, 8 pregnant mice. The number of viable fetuses was 48, the number of absorbed fetuses was 20, and the embryo resorption rate was 29.4%.

In the treatment group with a resveratrol dose of 25 mg/kg/d, there were 8 mice and 8 pregnant mice. The number of viable fetuses was 53, the number of absorbed fetuses was 10, and the embryo resorption rate was 15.9%.

In the treatment group with a resveratrol dose of 50 mg/kg/d, there were 8 mice and 8 pregnant mice. The number of viable fetuses was 60, the number of absorbed fetuses was 5, and the embryo resorption rate was 7.7%.

In the treatment group with a resveratrol dose of 100 mg/kg/d, there were 8 mice and 8 pregnant mice. The number of viable fetuses was 55, the number of absorbed fetuses was 14, and the embryo resorption rate was 20.3%.

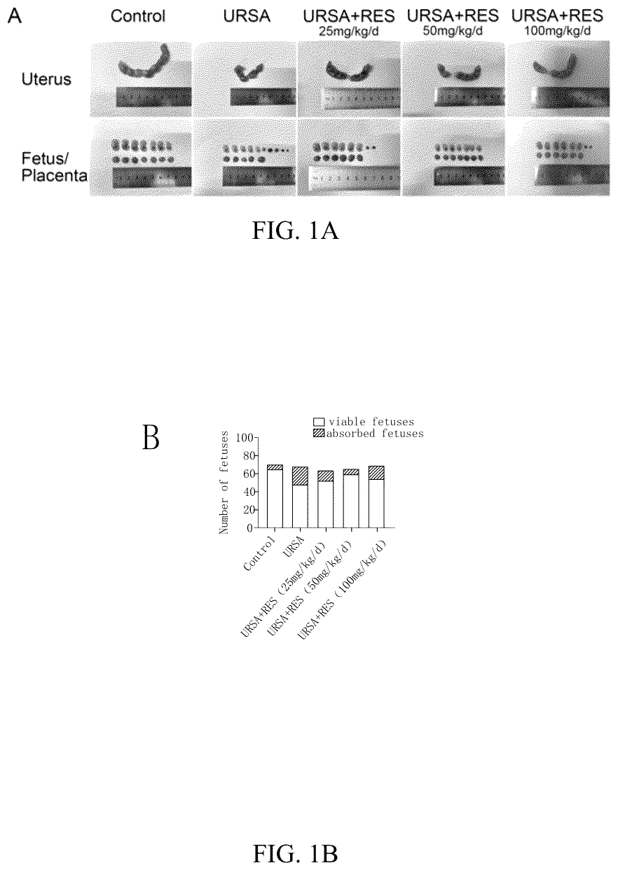

FIG. 1A and FIG. 1B showed the conditions of the uterus and placenta/fetus in the control group, model group, and drug intervention groups at doses of 25, 50, and 100 mg/kg/d. Among them, the embryos of mice were of basically the same size and rosy in color in the control group. In the model group, some embryos and placentas were smaller, or even no embryos were formed. Fetal resorption was less common in the three treatment groups. Compared with the control group, the embryo resorption rate in the model group was 29.4%. Compared with the model group, after treatment with resveratrol, the embryo resorption rates of the three treatment groups decreased significantly, reaching 15.9%, 7.7% and 20.3% respectively. Among them, the embryo resorption rate of the resveratrol treatment group with a dosage of 50 mg/kg/d decreased most significantly; so the group was chosen as the treatment group in subsequent experiments.

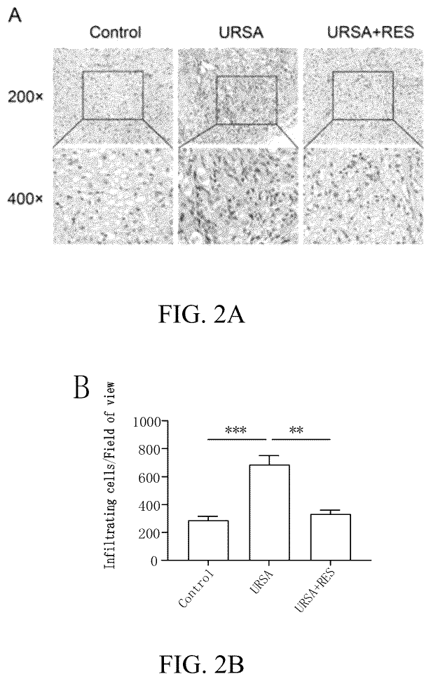

As shown in FIG. 2A and FIG. 2B, there were more immune cells infiltrating at the maternal-fetal interface of URSA mice. Among them, the number of decidual infiltrating cells in the model group was significantly higher than that in the control group. In the decidual tissues, the cells in the URSA group were densely packed, irregularly arranged, and had diffusely fragmented cell nuclei compared with the control group. However, the treatment group that was given resveratrol intervention showed significant improvement.

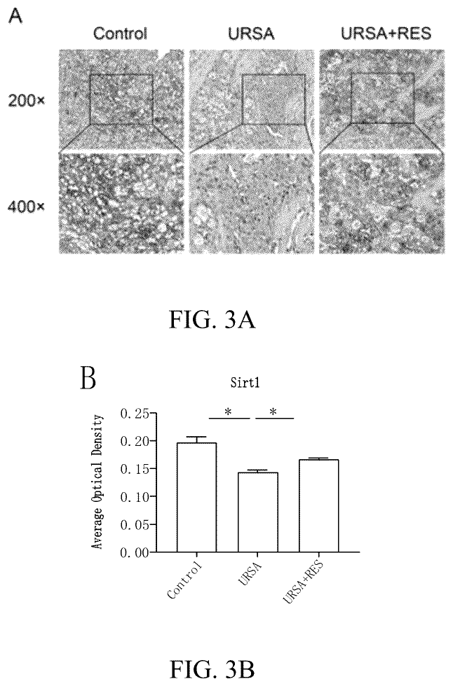

As shown in FIG. 3A and FIG. 3B, there were differences in the expression of Sirt1 protein in the decidual tissues of the three groups of mice. Among them, the expression of Sirt1 in the decidual tissues of the model group mice was significantly decreased as compared with that of the control group (brown area), while the expression of Sirt1 increased after treatment with resveratrol.

The above results showed that, in the mouse models of unexplained recurrent spontaneous abortion, the decidual tissue cells at the maternal-fetal interface were arranged disorderly, with a large number of immune cells infiltrating, and the expression of Sirt1 protein was decreased. After treatment with resveratrol (RES), the immune microenvironment at the maternal-fetal interface and the expression of Sirt1 protein were improved, which effectively demonstrated that Sirt1 could be used as a target for the prevention, protection and treatment of URSA disease by resveratrol and provide a theoretical basis for the prevention and treatment of clinical patients with URSA.

The foregoing description merely describes the preferred embodiments of the present disclosure and is not intended to limit the scope of the present disclosure. It should be noted that for those of ordinary skill in the art, several improvements and modifications can be made without departing from the design spirit of the present disclosure, and these improvements and modifications shall also fall within the scope of protection of the present disclosure.

1. Use of resveratrol in the preparation of a product for preventing and/or treating unexplained recurrent spontaneous abortion.

2. The use according to claim 1, wherein the product is a drug.

3. The use according to claim 2, wherein the resveratrol is the only active ingredient in the drug.

4. The use according to claim 3, wherein the drug further contains medically acceptable excipients.

5. The use according to claim 4, wherein a dosage form of the drug is injection, solution, emulsion, suspension, suppository, ointment, cream, spray, drop, powder, granule, dissolved medicine, capsule, pill, tablet, patch or sustained-release preparation.

6. Use of a composition in the preparation of a product for preventing and/or treating unexplained recurrent spontaneous abortion, wherein the composition contains resveratrol.

7. The use according to claim 6, wherein the product is a drug.

8. The use according to claim 7, wherein the drug further contains medically acceptable excipients.

9. The use according to claim 8, wherein a dosage form of the drug is injection, solution, emulsion, suspension, suppository, ointment, cream, spray, drop, powder, granule, dissolved medicine, capsule, pill, tablet, patch or sustained-release preparation.
